SEQUENCE LISTING (10 sequences)

SEQ ID NO: 1 - DNA, 11 bp, Artificial Sequence
  Description of Artificial Sequence Synthetic oligonucleotide
ataaaaaaaag

SEQ ID NO: 2 - DNA, 11 bp, Artificial Sequence
  Description of Artificial Sequence Synthetic oligonucleotide
aataaaaaaag

SEQ ID NO: 3 - DNA, 11 bp, Artificial Sequence
  Description of Artificial Sequence Synthetic oligonucleotide
aaataaaaaag

SEQ ID NO: 4 - DNA, 11 bp, Artificial Sequence
  Description of Artificial Sequence Synthetic oligonucleotide
aaaaaaaaaag

SEQ ID NO: 5 - DNA, 11 bp, Artificial Sequence
  Description of Artificial Sequence Synthetic oligonucleotide
aagaaaaaaaa

SEQ ID NO: 6 - DNA, 11 bp, Artificial Sequence
  Description of Artificial Sequence Synthetic oligonucleotide
aaaaagaaaaa

SEQ ID NO: 7 - DNA, 10 bp, Artificial Sequence
  Description of Artificial Sequence Synthetic probe
gggatacccc

SEQ ID NO: 8 - DNA, 10 bp, Artificial Sequence
  Description of Artificial Sequence Synthetic probe
gggatatccc

SEQ ID NO: 9 - DNA, 10 bp, Artificial Sequence
  Description of Artificial Sequence Synthetic probe
ggggcttccc

SEQ ID NO: 10 - DNA, 10 bp, Artificial Sequence
  Description of Artificial Sequence Synthetic probe
ggggctcccc

(19) United States
(12) Patent Application Publication
(10) Pub. No.: US 2009/0305237 A1
(43) Pub. Date: Dec. 10, 2009

(54) QUANTIFICATION OF NUCLEIC ACIDS AND PROTEINS USING OLIGONUCLEOTIDE MASS TAGS

(75) Inventors: Charles R. Cantor, Del Mar, CA (US); Lingang Zhang, Natick, MA (US); Simon Kasif, Newton, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 11/914,970

(22) PCT Filed: May 26, 2006
(86) PCT No.: PCT/US06/20470

§ 371 Date: Jun. 16, 2008

Related U.S. Application Data
(60) Provisional application No. 60/684,746, filed on May 26, 2005.

(51) Int. Cl. C12Q 1/68

(52) U.S. Cl. 435/6

(57) ABSTRACT

The invention provides a method for detecting and quantifying the amount of target molecules, such as nucleic acids or proteins in a sample. The target molecules are first recognized and bounded by target-specific probes, generally nucleic acids or proteins that bind specifically to the targets, each of which is labeled with a short single-stranded nucleic acid probe, either DNA or RNA, with distinct molecular weight. This label is called an oligonucleotide mass tag. One or several standard oligonucleotide sequences can be designed with similar sequence but distinct molecular weight to those oligonucleotide mass tags. Then the oligonucleotide mass tags associated with bounded probes and the standard sequences are co-amplified using a pair of common primers. The presence and/or amount of each oligonucleotide mass tag, which corresponds to the amount of corresponding target molecule, is determined by a primer extension reaction and quantification of the primer extension product.

### CROSS-REFERENCE TO RELATED APPLICATIONS

This application claims the benefit under 35 U.S.C. §119(e) of the U.S. provisional application Ser. No. 60/684,746, filed May 26, 2005, the content of which is herein incorporated by reference in its entirety.

### BACKGROUND OF THE INVENTION

Quantitative detection of nucleic acids, proteins and nucleic acid protein interaction specificities is of crucial importance to biomedical research and clinical applications. For example, detection and quantification of differentially expressed genes and their splicing variants in a number of pathological conditions would be useful in the diagnosis, prognosis and treatment of these pathological conditions. Quantification of gene expression and proteins would also be useful in diagnosis of infectious diseases and following up effects of pharmaceuticals or toxins on molecular level. Quantification of nucleic acid protein interaction specificities provides insight into the regulation of cell growth and differentiation.

The rapid pace of discovery of new genes generated by large-scale genomic and proteomic initiatives has required the development of high-throughput strategies to quantify the expression of a large number of genes and their alternatively spliced isoforms, as well as elucidate their biological functions, regulations and interactions (1, 2). A number of high-throughput techniques have been developed to detect and quantify nucleic acids, proteins and nucleic acid protein binding specificities.

Two basic categories of high-throughput techniques are widely used to study nucleic acids. One is microarray-based analysis. Microarrays, including cDNA and oligonucleotide microarrays, are capable of profiling expression of thousands of genes in a single experiment (3). They have been widely used in gene expression profiling (gene array) (4), alternative splicing analysis (exon or exon-junction array) (5-10), and transcript annotation (genome tiling array) (11-13). The other approach involves the sequencing of a short tag of each transcript. This mainly includes Expressed Sequence Tag (EST) sequencing (2) and Serial Analysis of Gene Expression (SAGE) (14). Because each tag is likely to uniquely correspond to a single gene or exon, the number of each tag could be used to estimate the expression level of the corresponding genes or alternatively spliced variants, or to identify novel genes or exons.

However, both microarray and tag-sequencing techniques are not sensitive and demand relatively high input levels of mRNA that are often unavailable, particularly when studying human diseases. With the amplification of RNA products using RT-PCR, it is possible to use substantially less starting material (15, 16). However, the amplified population may not faithfully represent the original RNA population because of inherent non-linear nature of the PGR reaction and amplification bias between samples (17).

In addition, for microarray study, the array quality is often a problem for cDNA or oligonucleotide microarrays. For example, most researchers cannot confirm the identity of what is immobilized on the surface of a microarray and generally have limited capacity to check and control possible errors in the microarray fabrication. Also, microarray analyses provide only relative expression levels in a carefully controlled experiment. The lack of absolute quantification makes it difficult to compare results from different experiments or different arrays. Additionally, the high costs of microarrays have caused many investigators to perform relatively few control experiments to assess the reliability, validity, and repeatability of their findings. Moreover, in microarray experiments, fluorescence labeling is generally used. In addition to the increased experimental complexity, after hybridization the intensity of the fluorescence on each spots on the array may not be well correlated with the amount of corresponding mRNA.

For the tag-sequencing analysis, a large amount of sequencing effort, generally slow and costly, is needed for tag-based analysis and the sensitivity of tag-based analyses is relatively low and high sensitivity can only be achieved by sequencing a large number of tag sequences.

Another method to analyze gene expression using a standard competitor and competitive PGR was recently developed by Ding and Cantor (United States Patent Application 20040081993) (18). In this method absolute quantification of transcript copy numbers in a sample can be determined by Mass Spectroscopy. Typically, the standard sequence of about 80 bases is needed for each target of interest. In addition, the throughput is limited by the multiplexity of PGR amplification.

There are primarily two approaches to characterize multiple proteins in biological samples: 2D-gels and protein microarray. The 2D-gel approach is both time-consuming and unsuitable for the analysis of low abundant protein (19). Although high-throughput protein profiling has been demonstrated using protein microarray (20-38), different problems inherent in its methodology are limiting its wider applications (33). For example, the sensitivity is generally low. In addition, proteins of interest are generally labeled with fluorescence before the differential-capture microarray assays, and one has to be aware that proteins are often assembled into multi-protein complexes. Therefore, a strong signal can either result from a large amount of captured target protein or from the capture of a large complex of different proteins. Methods have been developed to alleviate some of these problems. For example, immuno-PCR achieved very high sensitivity of protein detection (39), however, it appears difficult to run in a high-throughput, multiplexing fashion.

Nucleic acid-protein interactions play an important role in regulating many biological processes, including cell growth, survival and differentiation. For instance, transcription factor (TF) and DNA binding regulates the transcription of RNA. The availability of genomic sequences underscores the importance of in vitro nucleic acid-protein interaction assay to discover protein-binding sites in the genome and elucidate how mutations and polymorphisms in the protein-binding sites impact the binding affinity and thus the transcription regulation. Traditional technologies aimed at characterizing DNA-protein interactions, including electrophoretic mobility shift assay (EMSA), are time-consuming and not scalable. The protein-binding microarray (PBM) technology allows the determination of in vitro binding specificities of individual transcription factors in a high-throughput fashion (42). However the assay requires complicated chemistry to fabricate the PBM and demands a large amount of purified target proteins. Additionally the heterogeneous interaction of TF and DNA probes immobilized on the solid surface is a concern.

Thus it would be desirable to develop methods which allow a sensitive and accurate quantification of nucleic acids or proteins, can be easily automated and scaled up to accommodate testing of large numbers of sample and overcome the problems associated with available techniques. Such a method would permit diagnosing different pathological conditions. The method can be used for diagnostic, prognostic and therapeutic purposes, and would facilitate genomic, pharmacogenomic and proteomic applications.

### SUMMARY OF THE INVENTION

The present invention provides a method of measuring the amount of target nucleic acids, proteins or analyzing nucleic acid protein binding specificities in a sample using a short single-stranded nucleic acid or nucleic acid analog sequence with distinct molecular weight, referred herein as “oligonucleotide mass tag” (OMT or massTag) to label each target-specific probe, usually a nucleic acid or protein.

The target is typically a nucleic acid or protein and the probe is typically a nucleic acid sequence or a nucleic acid sequence linked to a protein. After incubation of the probes with targets, the OMTs associated with those probes that bind to the targets are amplified using, for example, PGR reaction. The amount of each oligonucleotide mass tag is determined, for example, by a primer extension reaction followed by a quantification of the primer extension products. In one preferred embodiment, the quantification is performed using Mass Spectrometry.

Absolute quantification of the target sequence can be achieved by introducing one or several standard oligonucleotides with known sequence and known concentrations as competitors in the PGR reaction. The standards are designed to be similar in length and sequence to the target sequence, but distinct in molecular weight from the detectable oligonucleotide mass tags associated with the target identifying probes.

Accordingly, in one embodiment, the invention provides a method for gene expression analysis comprising the steps of designing two 5′-3′ probes, wherein the 5′ end of the probe 1 comprises region A, a nucleic acid sequence detected by a primer 1, region B comprises a massTag sequence, and region C, the most 3′-region of the probe 1 comprises the target recognition sequence, that will bind the target molecule. Probe 2, comprises in its most 5′-region, the target recognizing sequence (region D), then the massTag (region E), and in its most 3′-region, a constant region recognized by primer 2 (region F). A double-stranded target nucleic acid and the probes are then combined and the regions C and D of probes 1 and 2, respectively, anneal to the target. The probes are ligated together, using a ligase enzyme, for example, T4 ligase, and thus form a continuous nucleic acid sequence comprising 5′-A-B-C-D-E-F-3′. Primers are designed to anneal to regions A and F, and an amplification reaction can consequently be performed. After amplification, the excessive dNTPs are removed, for example, using alkaline phosphatase, and a primer extension reaction is performed using a primer with the sequence of region A. The OMTs (region B) are designed to comprise a specific base (herein called termination base) at the most 3′ end and three other bases elsewhere. Using a proper combination of dNTP/ddNTP in the primer extension (herein referred as extension mix), where the ddNTP is the dideoxy-form of the termination base and the dNTPs comprise the other three bases, the primer extension will stop at the 3′ end of the OMTs (region B). The primer-extension products, each of which comprises 5′-A-B-3′ and therefore has a unique mass, can be quantified, preferably using a mass spectrometer. Because different targets are designed with differentiable OMTs in region B, different primer-extension products can be resolved and quantified simultaneously in a multiplexing fashion using a mass spectrometer. In one embodiment, the primer extension primer anneals to either A or F region or both and extension is performed so that it ends at the beginning of the C or end of the D region so as to amplify the massTag in its entirety. The primer extension products are analyzed to quantify the amount of them in the sample, preferably using MS. In another embodiment, a known amount of one or more control sequences is added to the amplification reaction to allow the absolute quantification of the OMTs and the comparison of quantification results from different experiments.

In another embodiment, the invention provides a method for protein expression analysis comprising the steps of designing a probe comprising at least four regions, region P in the most 5′-end of the probe, comprises a protein capture molecule, for example, an antibody or an aptamer, region A comprises a constant nucleic acid sequence recognized by primer 1, region B comprises the massTag sequence, and region C, a sequence recognized by primer 2. The proteins are extracted from the sample and immobilized to a solid surface before, after or simultaneously with combining the protein with the P-5′-A-B-C-3′-probe. For example, After washing up the unbound probes, the solid phase immobilized probes are served as templates in the consequent PCR3′-probe. The solid-phase-protein-probe combination is then used as a template in an amplification reaction. The excess nucleotides are removed, for example using an alkaline phosphatase enzyme, and a primer extension reaction is performed to allow extension of the massTag. The primer extension products are consequently analyzed, for example, using MS. In one embodiment, one can add at least one standard probe with a different massTag to the amplification reaction which allows absolute quantification of the primer extension products.

In yet another embodiment, the invention provides a method of quantifying nucleic acid-protein binding specificities. For instance, of transcription factor (TF) and DNA binding assay (schematically shown in FIG. 4A), a number of DNA probes are designed, each of which comprises four regions, e.g., A, B, C, and D from the 5′-end to 3′-end. Region A and D compromise constant sequences and are respectively recognized by the two PCR primers. Region B compromises OMT, and region C compromises the candidate TF-binding sequences. Each probe is annealed with equal molar of its reverse-complementary sequences to form double-stranded probe. Then the target TF (for example, purified TF protein or cell extract containing the target TF protein) is incubated with a mixture of different double-stranded probes and an antibody that is specific to the TF (herein referred as 1^{st} antibody). Then a certain amount of antibody-coated beads (for example, superparamagnetic Dynabeads) is added and incubated. The antibody immobilized on the beads, referred as the 2^{nd} antibody, binds specifically to the 1^{st} antibody. After washing up the beads, PCR amplification is performed using a common pair of primers and the probes immobilized on the beads serve as the templates. After PCR the excessive nucleotides are removed, for example, using an alkaline phosphatase enzyme, and a primer extension reaction is performed. The primer extension products, which compromises 5′-A-B-3′, are quantified, for example, using a mass spectrometer. In one embodiment, one can add at least one standard probe with a distinct OMT to the PCR mixture for co-amplification, which allows the absolute quantification of OMTs and the comparison of quantification results from different experiments.

In yet another embodiment, the invention provides a method for analyzing protein-protein interactions. For each protein, a distinct single-stranded oligonucleotide label is designed and synthesized. Each label consists of 3 regions, named from 5′ to 3′ A1, A2, and A3 for protein A and B1, B2, and B3 for protein B. Region A1 and B1 are 15˜20 bases, and A2 and B2 are about 5˜30 bases, and A3 and B3 are about 10 bases. Protein A and B can be directly linked to their corresponding single-stranded oligonucleotide labels using well established procedures (see, for example, references 32, 40, 41). Alternatively the linkage can be achieved by introducing protein-specific aptamer at the 5′ end of each oligonucleotide label in the label synthesis step. The labeled protein A and B are mixed together with the connector, together with the T4 DNA ligase and its buffer. If protein A and B interact with each other, then region A3 and B3 can be ligated together because of proximity (42). After ligation, PCR primer A1 (same to region A1) and B1′ (antisense to region B1) are added and an amplification reaction, such as PCR is run using standard protocols (see, e.g., reference 18). After the amplification, excess dNTP are removed, for example using SAP or other alkaline phosphatases well known to one skilled in the art. Then, primer A1 is added together with dATP, dCTP, dTTP, ddGTP, and ThermoSequenase (SEQUENOM®). Then the primer extension is conducted following standard protocols (see, e.g., reference 18). The primer extension will stop at the 3′ end of region A2. The amount of sequences A1A2 can be quantitatively detected using the surface area of the signal peak of A1A2, for example using mass spectrometry (MS). The amount of A1A2 reflects the amount of interacted proteins in the sample. Thus, one can calculate the amount of the initial target.

The method of the present invention uses nucleic acid sequences with distinct molecular weight as labels. The labels can be efficiently amplified, for example, using PCR reaction so that targets with very low copy numbers can be sensitively detected. Using a pair of common primers for PCR amplification and a common primer for primer extension, the method is not only inexpensive, but also efficient and can be applied in a multiplexing fashion for high-throughput analysis with virtually no optimization for amplification when using for example PCR.

In addition to the use of common primers for amplification, the length and sequences of the amplification, such as PCR, targets, oligonucleotide mass tags and standards, are designed to be similar to each other so that the amplification efficiency for all amplification targets is also very close. As a result the amplification process preserves faithfully the ratio of the standard and the oligonucleotide mass tags associated with the target(s) of interest. Thus, absolute quantification can be achieved and results from different experiments are comparable.

By using nucleic acid amplification, one can quantify targets, including nucleic acids and proteins, of very low abundance thus providing a very sensitive detection system. Therefore the invention can be applied to diagnosis and study of diseases. For example, diagnosis of diseases that are caused by reduced amount of gene produce is one useful application. In many of these diseases the copy number of mRNAs produced from a defective allele is low, and thus one can easily miss detecting the disease causing mutation if one studies the sequence of the transcript using the traditional amplification and sequencing protocols. The method also allows a sensitive protein detection/quantification method for, for example diagnostic purposes, from biological samples. Methods of the invention can thus be used also in the diagnosis of microbial, parasitic, and viral diseases. The methods can also be used to diagnose and follow up disease conditions, that present themselves with secretion or leaking of proteins in urine or other biological fluids where they are normally not present. Similarly, any protein or nucleic acid, can be detected from any organic or inorganic sample using the presently described methods.

One can also combine the methods of the present invention with immuno-PCR or other amplification methods.

The method also allows absolute quantification of the target molecules. In the method of present invention, the length and sequence of all the amplification targets (oligonucleotide mass tags and standards) are very similar. Therefore, the amplification efficiency for different targets is practically identical. As a result, the amplification process faithfully preserves the ratio of the nucleic acid standard and the target nucleic acid of interest. Thus, results from different experiments should be comparable. Using the standards with known concentration as competitors in the amplification reaction, absolute quantification can therefore be achieved.

In the methods of the invention, a pair of common amplification and extension primers can be used for all oligo massTags as well as the standards for amplification and for all primer extension reactions. The strong resolving power of mass spectrometry (MS) can detect a large number of primer-extension products. As a result, this method can be easily used in a high throughput way. For example, using a multiplicity of only 10 and the typical 384-microwell format SPECTROCHIP™ (SEQUENOM), we could analyze 3840 targets on one chip.

The present method is superior to methods using electrophoresis in detection, wherein the detection relies on size difference in the tag. The method allows accurate detection of tags that have the same size, and that only differ slightly in the sequence of the massTag. This is particularly advantageous when one uses PCR to amplify the targets and controls because even a slight difference in size between a target amplification and a control amplification may result in different amplification efficiency of the nucleic acid and thus inaccuracies in the detection of the absolute quantity of the initial target. Moreover, the capacity to detect and quantify multiple targets at the same time is significantly improved by the methods of the present invention.

The present method also provides a significant advantage in detection accuracy and multi-target detection capacity over detection methods that utilize array technology that employ biotin-streptavidin interactions as detection means.

The present method is also superior to methods using the traditional small organic compounds or simple fluorescent molecules as mass tags. The variety of different oligonucleotide tags that one can produce is practically limitless. Thus, the method allows detection and quantification of many more targets in a single reaction when compared to methods using traditional small organic molecules as mass tags. Moreover, targeting and ligation of nucleic acids to nucleic acids or proteins such as antibodies, are performed using very basic and routine techniques, and thus require no specific chemical reactions that are typically necessary when one wishes to add a small organic molecule as a detectable “tag.” Thus the present method significantly simplifies analysis of molecules, such as proteins and nucleic acids.

Typically, the oligonucleotide sequences that are used for the nucleic acid analysis in the methods of the present invention are synthesized using traditional oligonucleotide synthesis methods, and thus the quality is generally high, for example, generally better than, for example, the AFFYMETRIX, Inc., microarrays that use photo-directed in situ synthesis that is more prone to inefficiencies in production. In addition, the hybridization of probes and targets occurs in a homogeneous buffer, rather than the heterogeneous environments of microarrays. Thus, the hybridization reaction is typically more efficient and homogeneous in the methods of the present invention. As a result, more accurate and reproducible results are achieved.

Compared with the gene expression analysis method using a comparative PCR and MS, the current method has the advantages of, for example, high multiplicity that can be more easily achieved because a common pair of primers is used for all targets; and b) cost of oligonucleotide synthesis are reduced; and c) it is hard to implement competitive PCR to study alternative splicings close to the ends of the mRNA sequences (e.g. alternative splicings in the first or last exon) because of the lack of proper PCR-amplifiable region, or the insertion/deletion of very large exons because of the challenge of synthesizing very long standard sequences and the PCR bias of templates of significantly different lengths (43). Therefore, large scale profiling of gene expression is made not only possible, but also convenient and cost effective. In addition, in the traditional approach, a distinct long (about 80 bases) oligonucleotide competitor is synthesized for each target. The present method requires only one (or a mixture of several) common standards per reaction (not per target) that can be used for all targets. This approach significantly lowers diagnostic and experimental costs.

Moreover, because each oligo massTag represents a unique protein, protein complexes can readily and easily be resolved. This is significantly different from the typically used fluorescence labeling which often introduces errors when the protein is present in a complex.

### BRIEF DESCRIPTION OF THE FIGURES

FIG. 1 shows the number of differentiable oligonucleotide mass tags (OMTs) within specific length using four different terminators, ddATP, ddCTP, ddGTP and ddTTP. No two OMTs are within 16 Dalton of each other so that they can be accurately resolved and quantified in mass spectroscopy.

FIG. 2A shows a scheme of high-throughput gene expression analysis using oligonucleotide mass tags. FIG. 2B shows the mass spectrum of quantifying mRNA targets of Kanamycine and Luciferase. The labels ‘K’, ‘L’, and ‘primer’ indicate the peaks for Kanamycine, Luciferase and the extension primer respectively. FIG. 2C shows the relative quantification of mRNA targets, Kanamycine and Luciferase, at 5 different concentration ratio. The measured ratio is shown to be strongly consistent with the mRNA concentration ratio. The dots in the figure represents each of the 8 duplicate experiments and the line is the linear regression of the results.

FIG. 3 shows a scheme of high-throughput protein expression profiling using oligonucleotide mass tags.

FIG. 4A shows a scheme of protein/protein interaction analysis using oligonucleotide mass tags. FIG. 4B shows results of the 4-plex OMT assay of TF (NF-κB P50)-DNA binding specificity vs. the results of gel shift assay. The OMT results using four different PCR cycles are shown in the figure and the results are well consistent with the result from gel shift analysis (Pearson correlation coefficients >0.98). FIG. 4C shows results of the 10-plex OMT assay of TF (NF-κB P50)-DNA binding specificity vs. the results of gel shift assay. The two results are well consistent (Pearson correlation coefficient 0.825).

FIG. 5 shows the theoretical number of differentiable oligonucleotide mass tags of each length or up to each length from 5 bases. With a length from 5 to 30 bases, there are more than 40,000 differentiable oligonucleotide mass tags. Limited by the resolving capacity of the currently available detection technology, such as MS, this number may be practically smaller than the theoretical estimate, but is still large enough to provide potential labels for a large variety of different biological features.

FIG. 6 shows a scheme of high-throughput gene expression analysis using oligonucleotide mass tags.

FIG. 7 shows a scheme of high-throughput protein expression profiling using oligonucleotide mass tags.

FIG. 8 shows a scheme of protein/protein interaction analysis using oligonucleotide mass tags.

### DESCRIPTION OF THE INVENTION

The present invention discloses methods for measuring the amount of a target molecule, such as nucleic acid or protein, in a sample, or analyzing protein DNA binding specificities. This approach combines labeling using a target-specific probe, i.e., oligonucleotide mass tags (oligo massTags), simultaneous amplification (for example PCR, polymerase chain reaction) of the oligo massTags and standards, and primer extension, followed by mass spectrometric (MS) detection. As shown in the examples, slightly different procedures (i.e., modifications) are used for different applications, however, the general principle is the same. The method can be used for directly measuring copy numbers of target molecules in a sample, or comparing relative increase or decrease in the amount of the target molecules in different samples.

Accordingly, the method can be used for prognosis and diagnosis of diseases, wherein presence or absence of a molecule, such as nucleic acid with a particular sequence or a particular protein or a protein variant or a protein complex, is used as basis of the prognosis or diagnosis. The method can also be used in cases wherein detecting or following up levels of nucleic acids and/or proteins is required. The methods can also be used to analyze composition of protein complexes, for example, for diagnostic or drug development purposes.

As used herein, oligo massTags are short single-stranded nucleic acid sequences, either DNA or RNA, but usually DNA. Generally they are about 5, 6, 7, 8, 9, 10, 11, 12, 13, 14, 15, 16, 17, 18, 19, 20, 21, 22, 23, 24, 25, 26, 27, 28, 29, to 30 bases long. Each oligo massTag has a unique molecular weight, but can have many distinct DNA sequences. For instance, oligonucleotides sequences 5′-ATAAAAAAAAG-3′ (SEQ ID NO.:1), 5′-AATAAAAAAAG-3′ (SEQ ID NO.:2), and 5′-AAATAAAAAAG-3′ (SEQ ID NO.:3) are the same molecular weight. ‘AAAAAAAAAAG’ (SEQ ID NO.:4), ‘AAGAAAAAAAA’ (SEQ ID NO.:5), and ‘AAAAAGAAAAA’ (SEQ ID NO.:6) are also the same oligonucleotide massTag because they have the same molecular weight. Because of their distinct masses, different OMTs in a mixture can be simultaneously resolved and quantified, preferably using mass spectroscopy (18). Usually each OMTs are designed to comprise a base (herein referred termination base) at the most 3′ end and three other bases elsewhere. As a result the primer extension will stop at the 3′ end of the OMTs using the corresponding extension mix that comprise a combination of dNTP/ddNTP, where the ddNTP, referred as the terminator, is the dideoxy-form of the termination nucleotide and the dNTPs comprise the other three nucleotides. For instance, if the termination base is thymine (T), then in the primer-extension reaction the corresponding extension mix comprises dATP, dCTP, dGTP and ddTTP. For the purposes of the present invention, one prepares oligonucleotide sequence that have differences in their mass. Thus, because of their unique molecular weight, each oligo massTag can be quantitatively identified, preferably using mass spectrometry, such as matrix-assisted laser desorption/ionization time-of-flight (MALDI-TOF) mass spectroscopy (MS) (18).

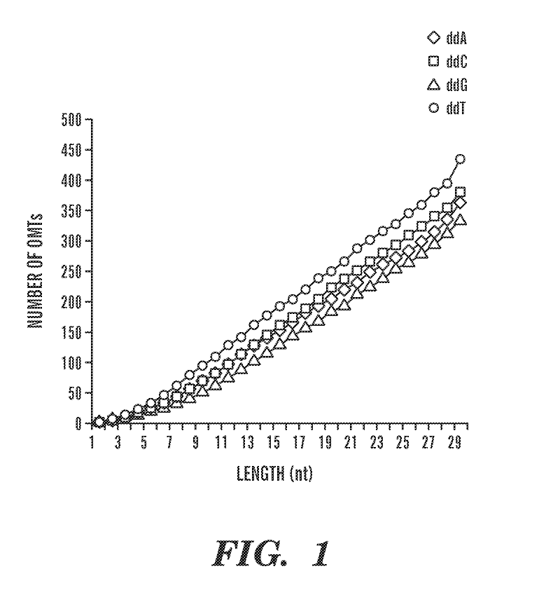

Generally mass spectrometer can resolve with a mass difference of at least 5 Daltons, for example, at least 10 Daltons . . . , 12 Daltons . . . , 15 Daltons . . . , 16 Daltons . . . , 20 Daltons. Thus, the OMT's should be made based upon this. Preferably, 16 or more Dalton (e.g. Sequenom's MassARRAY platform). As shown in FIG. 1, the number of differentiable OMTs (with a mass difference of at least 16 Dalton) within a length 30 of OMTs (length is 1˜30) can be over 400 using normal extension mix (dNTP and ddNTP) in the primer extension. As shown in FIG. 1, ddTTP is preferably used as the terminator to achieve maximal number of differentiable OMTs. Alternatively, mass-modified nucleotides may be used in the extension mix to generate more differentiable OMTs.

One of the major advantages of the present invention is that the number of differentiable oligo massTags is very large. As shown in FIG. 1, the number of differentiable oligo massTags, i.e., oligo massTags with different molecular weight, with a length from 5 to 30 bases is theoretically over 40,000. Presently, the resolving capacity of the currently available detection methodology, such as MS, the number of differentiable oligo massTags of each length is typically slightly less than the theoretical numbers in FIG. 1. However, with length from about 5 to about 30 bases, the total number of useful oligo massTags is still at the level of thousands, e.g. from about 10,000-40,000, 10,500-11,000-20,000, 25,000, 30,000 and up to approximately 40,000.

For example, a distinct OMT can be designed for and labeled to the probe (usually nucleic acid probe) for each target protein, mRNA, exon, or exon/exon junction like a zip code. After binding/annealing of the probe to the corresponding targets, the OMTs associated to the probes that bind/anneal to the targets can be PCR amplified, and then quantified after a primer extension reaction using mass spectrometry. The quantity of each target in the sample can be estimated from the peak area of the corresponding primer-extension product in the mass spectrum. The major advantages of OMT labeling includes, 1) it provides an amplifiable label, 2) different OMTs can label a large number of different targets in the same assay, and 3) OMT is generally a piece of nucleic acid sequence and can be designed and synthesized in the target-specific probe. No additional labeling process is required.

In one embodiment, a distinct OMT is first designed and synthesized in each target-specific nucleic acid probe. Each probe usually comprises at least three distinct functional regions, target-specific region for recognizing and binding the target molecule, OMT region for providing differentiable mass signal in the mass spectroscopy, and two constant regions recognized by PCR primers for PCR amplification. However the design of the probes can be slightly different for different applications, as shown in the three application examples. Generally, if the target is nucleic acid sequence, the target-specific region is generally nucleic acid sequences that are specific, e.g. reverse-complementary, to the target nucleic acid sequence. For quantification of proteins, the target-specific region can be protein (e.g. antibody) or nucleic acid (e.g. aptamer) that bind specifically to the target protein. For the protein-DNA binding specificity assay, the target-specific region is usually a piece of protein-binding consensus sequence.

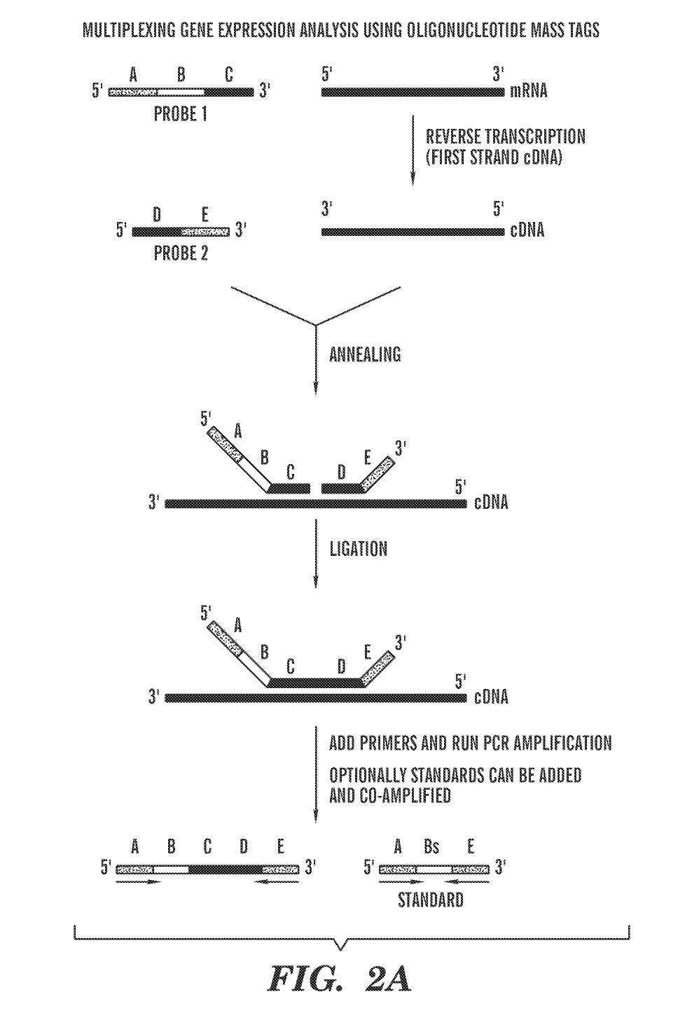

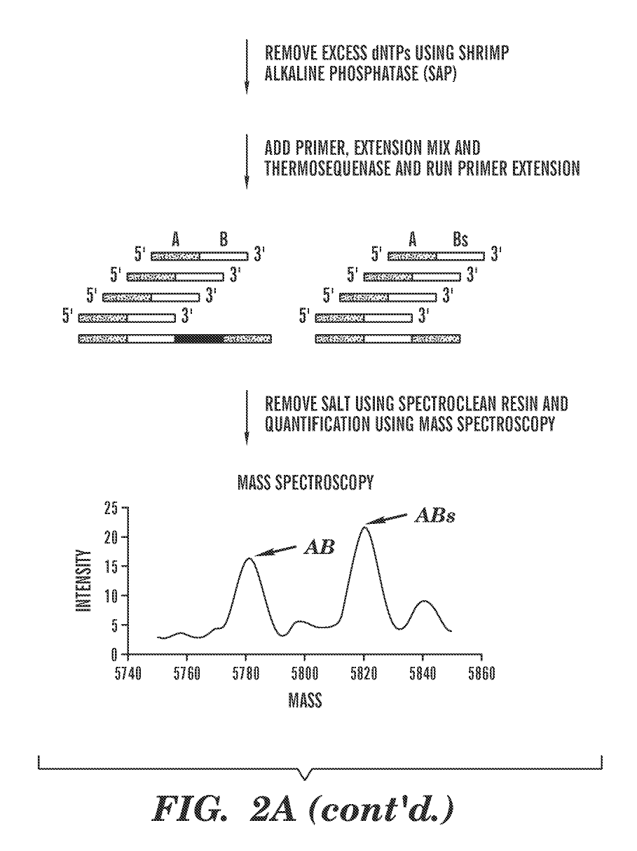

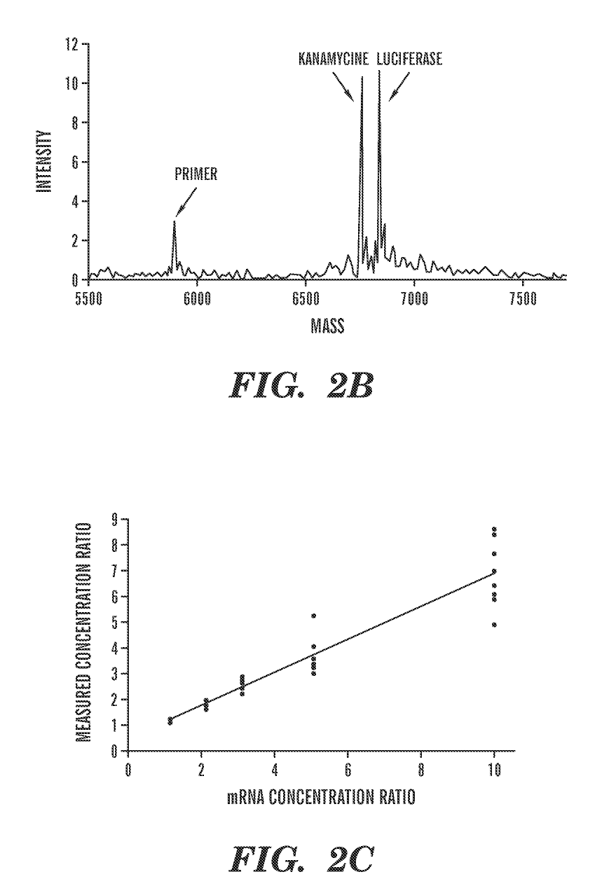

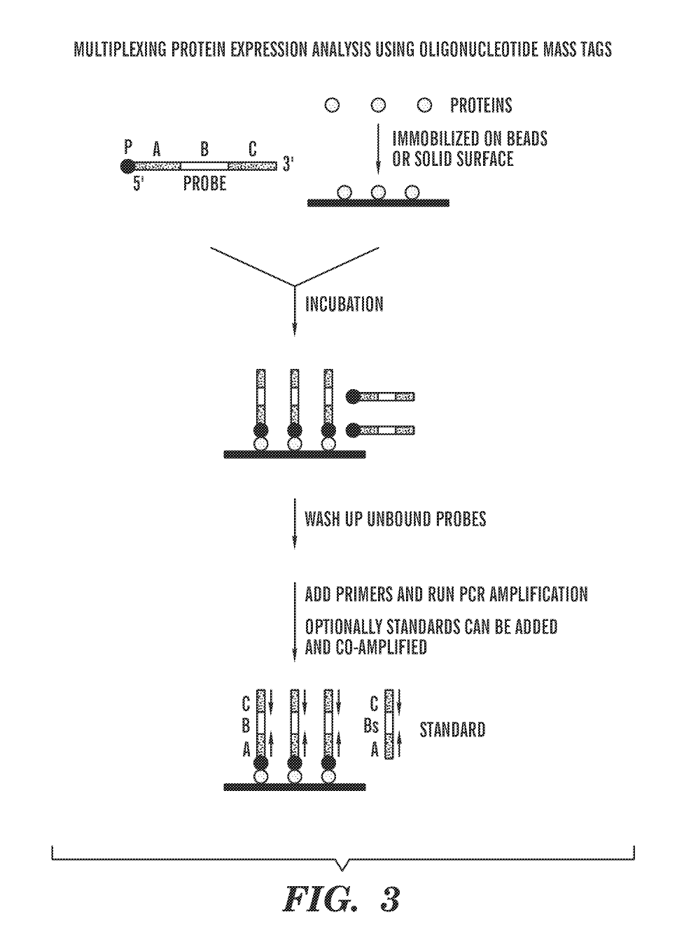

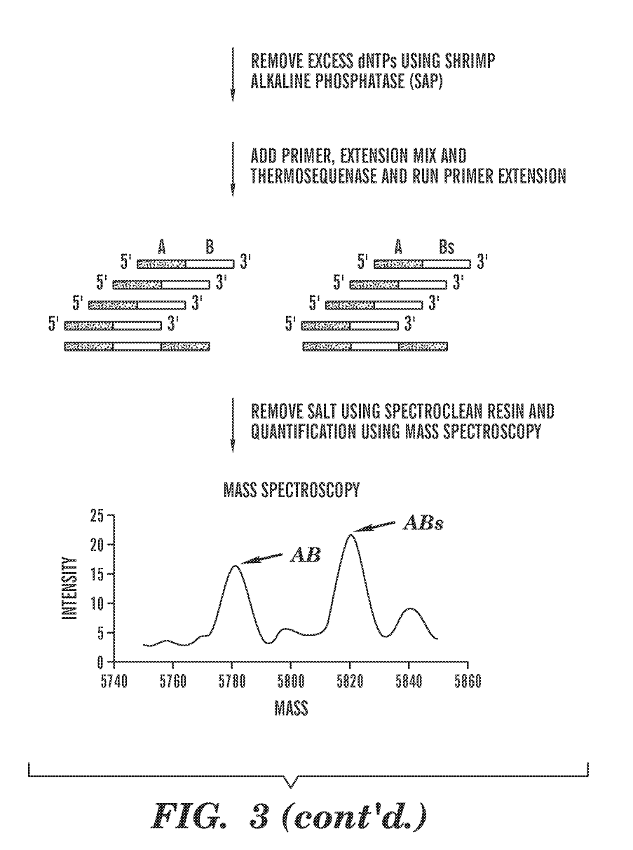

After incubation of the probes with target molecules, OMTs associated with probes that bind/anneal to the targets are PCR amplified using a common pair of primers. For absolute quantification, at least one standard nucleic acid sequences, which at least contain distinct OMTs but the same constant regions recognized by PCR primers, are also added at known concentration and co-amplified. As shown in the application examples, the OMTs associated with probes unbound to the targets won′ t be amplified because they are either washed up (e.g. FIG. 3, 4) or do not form a continuous template for PCR amplification (e.g. FIG. 2). After PCR, a primer extension reaction is performed using a common primer. Because the extension mix comprises a mixture of ddNTP of the termination base and dNTP of three other bases, single-stranded sequences comprising the OMTs and a constant primer sequence are generated as the extension products. Each of these extension products has a distinct mass and can be quantified in the mass spectra.

The large number of differentiable oligo massTags provides an efficient way to label different biological molecules or fragments thereof, including nucleic acids, proteins, and others, and then detect them using a simple set of reagents and conditions according to the methods of the present invention.

For example, a distinct oligo massTag can be uniquely assigned to avoid so problem of quantitatively detecting these biological features can be transformed to simply detecting the corresponding zip-code oligo massTags. These zip-code oligo massTags are amplified, and can be quantified, for example, using primer extension and, for example, mass spectroscopy analysis in a single reaction or in a high-throughput format.

In the present invention, a distinct oligo massTag is first designed and synthesized for each of the targets of interest, including proteins and nucleic acids. One also designs, preferably one common or constant oligonucleotide sequence per detection reaction (not per target) that will be added to each of the different target-specific massTags and that allows an amplification reaction with only one pair of primers for all the target that need to be detected from the sample. The oligo massTags, together with the constant oligonucleotides (used for amplification), are used to label their corresponding target-specific probes.

Thus each labeled probe generally consists of three portions, target-specific probe, oligo massTag that is unique to the probe, and constant oligonucleotide sequence for application reaction, such as PCR or an INVADER® assay.

For nucleic acid analysis, the target-specific probes are generally nucleic acid sequences that are specific, e.g. antisense, to the target nucleic acid sequences. For protein analysis, the target-specific probes that specifically bind to the target proteins can be proteins, e.g. antibodies, or aptamers, single-stranded oligonucleotides that assume a specific, sequence-dependent shape and bind to a target protein based on a lock-and-key fit between the two molecules. Such protein recognizing probes are well known to one skilled in the art.

After the two target-specific probes bind to the target molecule(s), they are ligated together, and the oligo massTags associated with the bounded probes are amplified, for example using a common pair of PCR primers.

For detecting whether a sample is positive or negative for any particular molecule, such as protein or nucleic acid, one can directly analyze the amplified products using MS. If the right sized signal is present, the target molecule is present in the sample. If one does not detect a peak of the size of the designed massTag, the target molecule was not present in the sample.

For absolute quantification, a standard or control nucleic acid which is similar in sequence otherwise but distinct in molecular weight to the oligo massTag, is also added and co-amplified. If one needs to have more than one molecule quantified, a different standard or control are added. Oligo massTags associated with unbounded probes will not be amplified because they lack proper primers (and are thus “cleaned up”), as shown in the figures and examples. The difference in the molecular weight among oligo massTags and the standards are then enhanced, for example, by a primer extension reaction using a common extension primer that anneals next to a differing nucleotide in the target and standard. The oligo massTags and standards are designed to use a combination of dNTP/ddNTP so that the primer extension will stop at specific, expected positions. The amount of each primer extension products, which reflects the amount of corresponding target, can be quantitatively determined by MS.

One can analyze any sample for presence or quantity of molecules such as nucleic acids, including double and single stranded RNA and DNA, and proteins using the method of the present invention.

A sample can be any sample including biological samples, such as blood, sputum, urine, saliva, tears, hair follicles, stool, spinal fluid, bone marrow, and any number of solid tissue or cell samples. The sample can also be a sample of non-animal or human origin, such as food, soil, water or a swiping of any material, wherein one wishes to detect presence or quantity of a particular molecule, such as nucleic acid or protein.

In the construction of the oligonucleotide primers, massTags and other nucleic acid reagents used in the methods of the present invention, one can also use well known nucleic acid analogs to improve, for example shelf life of the reagents. Naturally, if one uses oligonucleotide analogs in the templates that are intended to be amplified or synthesized in the primer extension reactions, one should choose the polymerase and sequenase enzymes so that they are capable of amplifying a template with the specific nucleic acid analogs. Such enzymes and optimization protocols are well known to one skilled in the art.

In one embodiment, A method for detecting a target nucleic acid in a sample comprising the steps of a) designing two 5′-3′ probes, probe 1 and probe 2, wherein the 5′ end of the probe 1 comprises a region A that is a nucleic acid sequence wherein a primer 1 anneals, a region B that comprises a massTag sequence that has a known unique mass, and a region C that is the most 3′-region of the probe 1 and comprises the target recognition sequence that binds the target molecule, and the probe 2 comprises in its most 5′-region a region D that is the target recognizing sequence, then a region E that comprises a massTag, and a region F in its most 3′-region that is a constant region recognized by primer 2; b) combining the target nucleic acid in a double-stranded form with the probe 1 and the probe 2, wherein the region C and the region D of the probes 1 and 2, respectively, anneal to the target; c) ligating the probe 1 and the probe 2 together, wherein the ligated product consists of a continuous nucleic acid sequence comprising 5′-A-B-C-D-E-F-3′; d) amplifying the 5′-A-B-C-D-E-F-3′ using the primer 1 and primer 2; e) optionally removing the excess dNTPs; and f) detecting the amplified product. In one embodiment, the method further comprises a step of quantifying the amount of the 5′-A-B-C-D-E-F-3′. In yet another embodiment, the method further comprises a step of quantifying the amount of the 5′-A-B-C-D-E-F-3′, wherein the quantification is performed using a primer extension reaction using a primer extension primer that anneals to either A or F region or both and wherein the primer extension reaction is performed so that it ends at the beginning of the region C or end of the region D so as to amplify the massTag in its entirety and analyzing the primer extension products.

In another embodiment, the method further comprises a step of quantifying the amount of the 5′-A-B-C-D-E-F-3′ wherein the quantification is performed using a primer extension reaction using a primer extension primer that anneals to either A or F region or both and wherein the primer extension reaction is performed so that it ends at the beginning of the region C or end of the region D so as to amplify the massTag in its entirety and analyzing the primer extension products the detecting in step f) is performed using mass spectrometry.

In one embodiment the method further comprises a step of quantifying the amount of the 5′-A-B-C-D-E-F-3′ wherein the quantification is performed using a primer extension reaction using a primer extension primer that anneals to either A or F region or both and wherein the primer extension reaction is performed so that it ends at the beginning of the region C or end of the region D so as to amplify the massTag in its entirety and analyzing the primer extension products, wherein a known amount of one or more standard sequences is added to the amplification reaction, wherein the standard sequences comprise a sequence otherwise identical to 5′-A-B-C-D-E-F-3′ except that the massTag region is designed to have a difference in its mass compared to the template 5′-A-B-C-D-E-F-3′, and wherein the primer extension reaction of the template and the standard allows determination of the absolute amount of target in the original sample.

In another embodiment, the invention provides a method for detecting or quantifying a target protein in a sample comprising the steps of a) designing a probe comprising at least four regions, a region P in the most 5′-end of the probe that comprises a protein capture molecule specific to the target protein, a region A that comprises a constant nucleic acid sequence recognized by a primer 1, a region B that comprises a massTag sequence, and a region C that comprises a sequence recognized by a primer 2; b) combining a sample suspected of containing the target protein with the P-5′-A-B-C-3′-probe; c) amplifying the 5′-A-B-C-3′ combination using the primer 1 and the primer 2; d) optionally removing excess nucleotides; e) performing a primer extension reaction wherein the massTag of the 5′-A-B-C-3′ is extended and results in a primer extension product if the target protein is present in the sample; and f) detecting the primer extension product. In one embodiment, the primer extension products is analyzed using mass spectrometry, preferably MALDI-TOF MS.

In one embodiment, the target protein is further quantified by, in addition to primer 1 and primer 2, adding to the amplification reaction a known amount of at least one standard oligonucleotide with otherwise identical sequence to 5′-A-B-C-3′ except for a different massTag, wherein after the primer extension reaction, if the target protein is present in the sample, a first primer extension product corresponding to the target protein and a second primer extension product corresponding to the standard oligonucleotide is produced, and wherein the primer extension products are detected using mass spectrometry and the ratio of the surface area that corresponds to the primer extension products with different mass indicates the amount of the target protein in the sample.

The invention also provides a method for detecting a protein-protein interaction comprising the steps of a) designing a unique single-stranded oligonucleotide label for each of the proteins or protein fragments that are involved or suspected to be involved in the protein-protein interaction, wherein each label consists of 3 regions, named from 5′ to 3′ a region A1, a region A2, and a region A3 for first protein or protein fragment (A), and a region B1, a region B2, and a region B3 for second protein or fragment (B) and so forth, and wherein the region A1 and the region B1 are 15-20 bases long, and the region A2 and the B2 are 5-30 bases long, and the region A3 and the region B3 are 10 bases long, and so forth; b) linking the protein or protein fragments that are involved or suspected to be involved in the protein-protein interaction to their corresponding single-stranded oligonucleotide labels; c) mixing the labeled proteins or protein fragments that are involved or suspected to be involved in the protein-protein interaction with a connector, together with a ligase, wherein the region A3, the region B3, and so forth, can be ligated together only if they are in sufficient proximity which occurs if the proteins or protein fragments interact with each other; and d) amplifying the ligated template using primer A1 that recognizes at least part of the region A1 and primer B1′ that is antisense to the region B1; e) optionally removing excess dNTPs; f) performing primer extension reaction, wherein the extension ends at the end of region 2 (A2 or B2′); g) detecting the primer extension products wherein presence of a primer extension product is indicative of protein-protein interaction between the proteins or protein fragments, and wherein absence of primer extension product is indicative of no interaction between the protein or protein fragments. In one embodiment, the analysis is performed using mass spectrometry, preferably MALDI-TOF MS.

In one embodiment, the method further comprised adding to the amplification reaction a known amount of at least one standard probe with sequence identical to each protein probe except for the massTag sequence, wherein the primer extension reaction of the template and the standard allows determination of the absolute amount of the protein or protein fragment in the protein complex.

There are a number of potential applications of oligo massTags for analyzing nucleic acids or proteins. For different applications the experimental procedures may be slightly different. However, the principle stays the same. As an example of analyzing nucleic acids, the experimental scheme for high-throughput gene expression analysis is presented in the following examples. However, the same procedure can be used for identifying alternatively spliced transcription isoforms, detecting exon/exon junctions, and discovering novel transcripts in the genome. For analyzing proteins, we demonstrate the schemes for protein expression profiling and protein/protein interaction analysis. However, the potential applications of the methods according to the present invention are by no means limited to these examples.

### Multiplexing Gene Expression Analysis

The experimental procedure for one preferred embodiment of a high-throughput gene expression analysis is schematically shown in FIG. 2A. The size of each region can be varied as is well understood by a skilled artisan, and is thus not limited to the size of the regions and probes in the examples. As shown in FIG. 2A, probe 1 comprises three regions that are A, B, and C from 5′- to 3′-end. Probe 2 comprises two regions, named D and E respectively from 5′- to 3′-end. Region A and E, constant in all probes, are generally 15˜25 bases long and are recognized by PCR primers for PCR amplification. Region C and D are about 10˜25 bases long and the concatenated sequence of region C and D, comprising 5′-C-D-3′, is reverse-complementary to the target of interest (e.g. the cDNA of a transcript). Region B is an OMT with a length of 1, 2, 3, 4, 5, 6, 7, 8, 9, 10, 11, 12, 13, 14, 15, 16, 17, 18, 19, 20, 21, 22, 23, 24, 25, 26, 27, 28, 29, to 30 bases. For each region C, a unique OMT region B is designed.

Synthesis of labeled probes is performed using standard oligonucleotide synthesis methods. For each transcript of interest, two oligonucleotide probes (Probe 1 and 2) are designed and synthesized using standard technique. Each probe has a length as described above. As shown in FIG. 2A, probe 1 consists of three regions that are A, B, and C from 5′ to 3′. Probe 2 also consists of 3 regions, named D, E, and F respectively from 5′ to 3′.

Region A and F, constant for all transcripts, are preferably about 5, 6, 7, 8, 9, 10, 11, 12, 13, 14, 15, 16, 17, 18, 19, 20, 21, 22, 23, 24, 25, 26, 27, 28, 29, or 30 bases. A skilled artisan can easily expand the size of the constant regions up to 40 or longer, 50 or longer, should there be need for longer sequences. In practice, this is rarely the case. One typically would use shorter or longer oligonucleotides to expand the selection of different massTags if the number of the targets in one reaction would be at the upper limit of diversity provided by the 5-30 nucleotide tags. Region C and D both are preferably about 10 to about 15 bases, for example, 10, 11, 12, 13, 14, or 15, long, but can also be made longer or shorter depending on the application. Such changes are well within one skilled in the art. For example C and D region can also be 3, 4, 5, 6, 7, 8, or 9 bases long, or alternatively 16, 17, 18, 19, or 20, bases long or even longer. The ligation of the 3′ end of region C with the 5′ end of D, called “CD”, is reverse-complementary to the target transcript (e.g., a gene, exon, or exon/exon boundaries). Region B and E, each of which has a length of 1, 2, 3, 4, 5, 6, 7, 8, 9, 10, 11, 12, 13, 14, to about 15 nucleotides, are two distinct oligonucleotide massTags with unique molecular weights. For each C and D, corresponding B and E are uniquely designed. For example, in region B it is a G (guanosine) at the 3′ end and A (adenosine), T (thymidine), and C (cytidine) elsewhere. In region E it is a C (cytidine) at the 5′ end and A (adenosine), T (thymidine), and G (guanosine) elsewhere.

To measure the amount of expressed transcripts in a sample, mRNA is preferably extracted for the target cell or tissue and used as target. Alternatively, first-strand cDNA is further synthesized using standard reverse transcription kits and used as targets. RNA extraction and reverse transcription can be performed using any specific methods well known to one skilled in the art.

Primer 1 and 2, DNA ligase, e.g. T4 DNA ligase or other ligase enzyme or mixture of ligase enzymes, as well as ligation buffers are mixed with the single-stranded cDNA. Generally, higher ligation temperature may increase the specificity of identifying target sequence. The optimization of hybridization conditions is routine to one skilled in the art. Ligation is preferably performed at higher temperature, e.g. 40-65° C., using thermostable ligase to improve the specificity. In the presence of the target sequence of interest, e.g., mRNA or cDNA, the 3′ end of Primer 1 and 5′ end of Primer 2 will be ligated together using the target sequence as a contactor as shown, for example, in FIG. 2A.

The ligation product, 5′-A-B-C-D-E-3′, is then PCR amplified using a pair of common primers annealed to region A and E. For absolute quantification, at least one standard sequence, which contains at least region A and E at the most 5′ and 3′ ends and a distinct OMTs contiguous to region A, is added at known concentration before the PCR reaction. There will be no PCR product in the absence of the target because of the lack of continuous PCR template. Amplification cycles (e.g., PCR reactions) of about 15-25 are generally performed.

During the amplification reaction, such as PCR, two amplification primers, A (the same to the sequence of region A) and F′ (antisense to the sequence of region F), DNA polymerase, dNTPs, as well as a standard oligonucleotide, or a mixture of several standard oligonucleotides, with known concentration as a competitor are added. Amplification using PCR can be performed using any number of cycles, for example, 15, 16, 17, 18, 19, 20, 21, 22, 23, 24, 25, 26, 27, 28, 30, 31, 32, 33, 34, 35, 36, 37, 38, 39 or 40, or even more cycles. The number of cycles can be determined by a skilled artisan and is not critical for the performance of the method. In one preferred embodiment, the PCR is run for about 15-25 cycles.

In all these procedures, one can follow, for example, standard homogeneous Mass Extension (MASSEXTEND®) assay protocols originally designed for the MASSARRAY® technology (SEQUENOM® INC.) or any other established, nucleic acid extraction, primer synthesis, ligation and amplification protocols. A skilled artisan can readily adapt any primer extension protocol for the use in the methods of the present invention.

In one embodiment, the standard sequence, like the concatenated primer 1 and 2, consists of region A, Bs, Cs, Ds, Es, and F. Regions Cs and Ds are of the similar length and sequences to the region C and D. Region Bs and Ds are oligo massTags with distinct molecular weight from those oligo massTags used for each target sequence.

After removing excess dNTPs from the amplification reaction, for example using alkaline phosphatase, such as Shrimp Alkaline Phosphatase (SAP), or other suitable enzyme, one adds primers A and F′ and three of the four deoxynucleotides and one of the four dideoxynucleotides, for example, on adds dATP, dCTP, dTTP, ddGTP, if a reaction is intended to end at the first G nucleotide, and a nucleic acid polymerase enzyme, such as sequenase, for example, THERMO SEQUENASE™ (Amersham). The primer extension can be conducted following any standard protocols, for example, protocols provided in Cantor et al. (ref. No. 18). As can be seen in FIG. 2A, because of the absence of ddGTP, the primer extension will stop at the 3′ ends of B and E′. At this point, one gets the extension product, 5′-A-B-3′. Because region A is constant sequence and B is OMT, the mass of different extension products can be solved by the mass spectrometer and therefore the amount of each primer extension product can be quantified by the peak area in the mass spectrum.

In a further step, the primer extension products, i.e. AB or F′E′ in the example of FIG. 2, can be quantified.

In one preferred embodiment, salts in the reaction buffer are removed from the final primer extension products. For example, the final primer extension products can be treated with SPECTROCLEAN™ (SEQUENOM, INC.) resin to remove salts in the reaction buffer using standard procedures (18). Other methods for removal of salts and/or buffers are known to one skilled in the art and can be used. Then the reaction solution is dispensed onto a mass spectrometer medium, such as a SPECTROCHIP™ (SEQUENOM, INC.) prespotted with a matrix of 3-hydroxypicolinic acid (3-HPA) by using a SpectroPoint (Sequenom) nanodispenser. A modified Biflex MALDI-TOF mass spectrometer (Bruker, Billerica, Mass.) was used for data acquisitions from the SPECTROCHIP. Other methods and systems for MS and analysis of MS data can easily be used in the spirit of the invention. The amount of sequences AB and F′E′ can be quantitatively detected using the signal ABs and F′Es′ as controls. Alternatively, in the primer extension step we can use only primer A and detect the amount of AB using MS. In this case, region E is unnecessary in primer 2. However, sometimes the amount of F′E′ can provide useful information as a control, and thus, in one embodiment, one uses both of the regions for quantification.

A mass spectrum of quantifying two mRNA targets, Kanamycin and Luciferase, is shown in FIG. 2B. Equal molar of the two mRNA are mixed at the total concentration of 0.3 nM and are then reverse-transcripted using random hexamer primers. The annealing and ligation are performed at >50° C. using Ligase 65 or Taq ligase, and PCR, SAP processing and primer extension are sequentially performed. As shown in FIG. 2B, the two extension products, labeled as K and L for targets Kanamycine and Luciferase respectively, are well resolved in the mass spectrum and can be quantified respectively using the MassTYPER (Sequenom) software.

In addition, Kanamycine and Luciferase mRNAs are mixed at five different ratios (i.e. 1:1, 1:2, 1:3, 1:5, 1:10) to a total concentration of 0.3 nM and used as the targets. Following the above experimental procedure, the area of each primer-extension product in the mass spectra is calculated using the MassTYPER software. The mRNA concentration ratio vs. the measured ratio (ratio of the peak area in the mass spectra) is well consistent, as shown in FIG. 2C. This result confirms that the OMTs can be well used for quantification analysis.

The amount of sequences AB reflects the amount of corresponding target in the sample. Thus, one can calculate the absolute amount of the initial target.

For example, the method of this example (FIG. 2A) can be directly employed to identify alternatively spliced variants by designing target-specific regions (CD) for a specific exon or exon/exon junction.

In addition, by designing target-specific regions specific to the genomic sequences, one can identify novel RNA transcripts, like in the genome tiling array.

High throughput protein expression profiling method is performed using oligo massTag, high-throughput protein expression analysis and it can be done by assigning a unique oligo massTag for each protein-capture probe, including, but not limited to antibodies, aptamers or others. The experimental procedure for protein detection and quantification using the methods of the present invention is schematically shown in FIG. 3.

For each protein-specific probe, a distinct single-stranded oligonucleotide label is designed and synthesized. Each label consist of at least 3 regions, named in this example as A, B, and C from 5′ to the 3′. Region A and C, constant for all the probes, are generally about 15, 16, 17, 18, 19, to about 20 bases long. Region B, about 1, 2, 3, 4, 5, 6, 7, 8, 9, 10, 11, 12, 13, 14, to about 15 bases long, has a distinct molecular weight and serves as the oligo massTag specific for the corresponding protein-capture molecule. In this example, we designed region B to have a guanosine (G) at the 3′ end and bases Adenosine (A), Cytidine (C), and Thymidine (T) elsewhere.

Each probe is labeled with its corresponding nucleic acid label. The protein-specific probes can be any molecules that recognize the protein or a fragment thereof, such as antibodies or aptamers. For antibodies, the labels can be linked to the antibodies using a variety of established procedures (see, e.g., references 32, 40, 41). For aptamers that bind specifically to the target proteins, the aptamer and the label are preferably synthesized as one nucleic sequence using standard nucleic acid synthesis technique (42). The labeled probes are preferably purified before using.

Proteins are extracted from cells and immobilized, for example, on micro beads or solid surface using established procedures (39). In one embodiment, the labeled probes are added and bind to the immobilized proteins. In another embodiment, the labeled probes and proteins are combined before binding the proteins on the solid surface. In yet another embodiment, binding of the protein on the solid surface is performed simultaneously with adding the labeled probe to the protein sample. Excess and unbounded probes are washed up.

Two amplification primers, such as PCR primers, A (same sequence to region A) and C′ (antisense to region C), DNA polymerase, dNTP, as well as a standard sequence, or a mixture of more than one standard sequences, with known concentration as a competitor are added and amplified. In one embodiment, the PCR amplification reaction is run for about 40 cycles using standard protocols (18). The standard sequence, like the concatenated primer 1 and 2, consists of region A, Bs, and C. Region Bs is oligo massTags with molecular weight different from those oligo massTags used for targets.

After the amplification, the excess dNTPs are preferably removed using SAP. Then, primer A is added together with dATP, dCTP, dTTP, ddGTP, and a sequenase, such as THERMOSEQUENASE (Amersham). The primer extension is conducted following standard protocols (18). The primer extension will stop at the 3′ end of region B because the region B is designed to have a base G at the 3′ end, and A, C, or T elsewhere. The primer extension generates the concatenated region A and B (called AB), which can further be quantified using the peak areas produced by MS of the sample and standard or control as a reference.

In one preferred embodiment, the quantification is performed using Liquid Dispensing and MALDI-TOF MS. In this embodiment, the final primer extension products are treated to remove salts in the reaction buffer using standard procedure (18), for example, with SPECTROCLEAN (SEQUENOM) resin. The reaction solution is dispensed onto a SPECTROCHIP (SEQUENOM) prespotted with a matrix of 3-hydroxypicolinic acid (3-HPA) by using a SPECTROPOINT (SEQUENOM) nanodispenser. A modified Biflex MALDI-TOF mass spectrometer (Bruker, Billerica, Mass.) was used for data acquisitions from the SPECTROCHIP. The amount of sequences AB can be quantitatively detected using the signal ABs as controls. The amount of AB reflects the amount of corresponding target proteins in the sample. Thus, the amount of the initial target can be calculated.

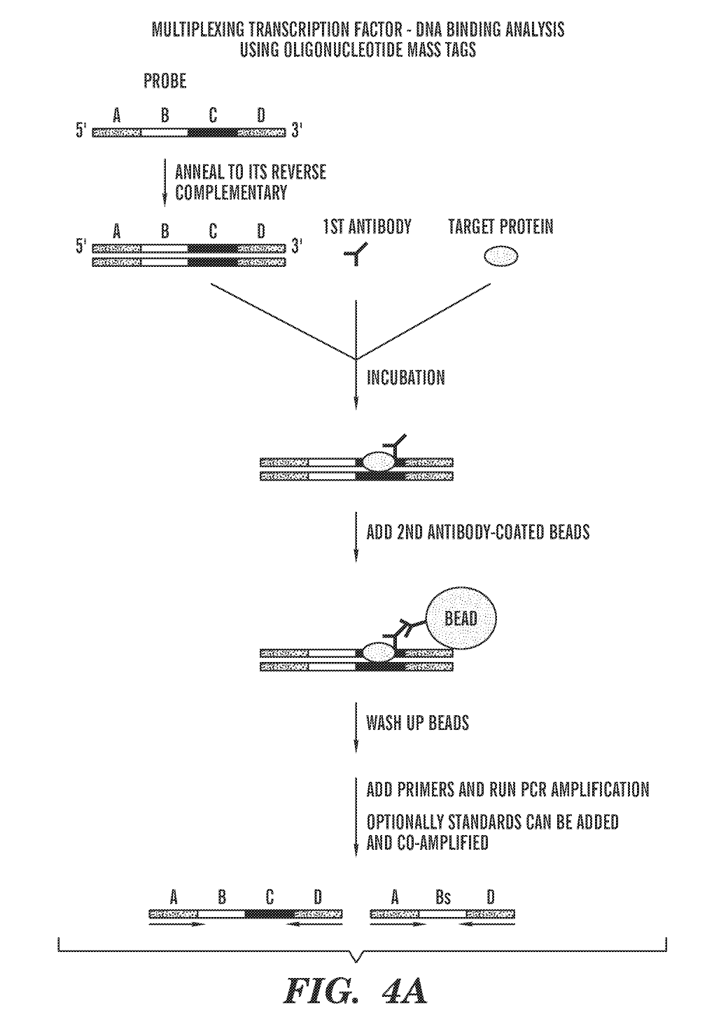

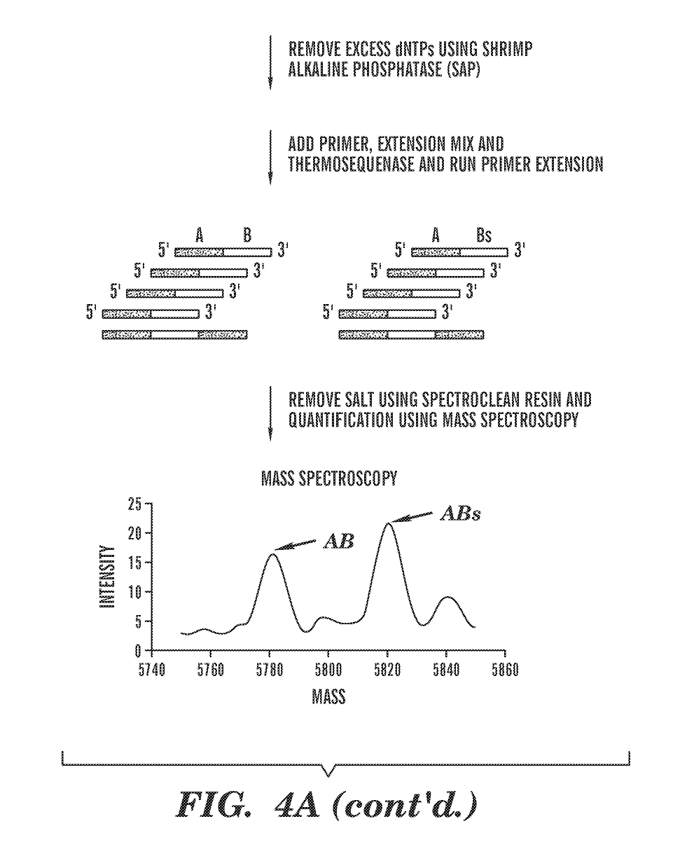

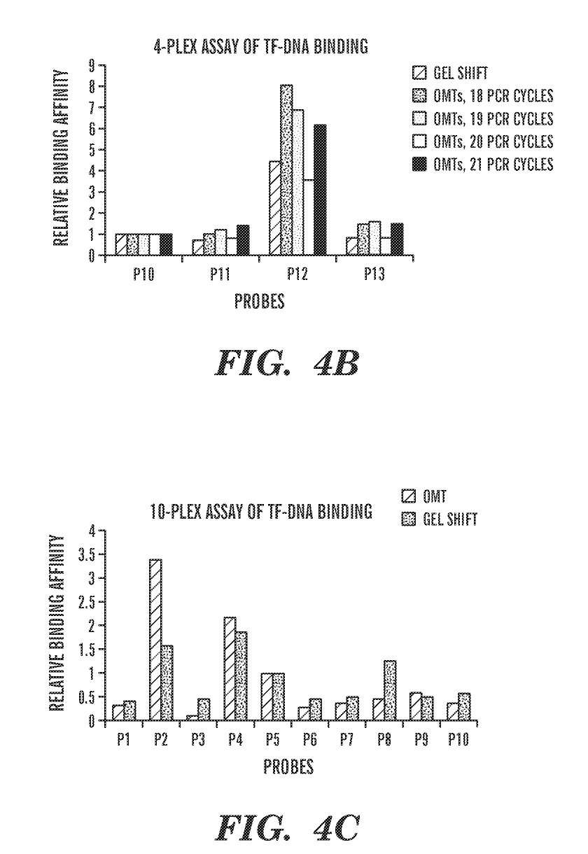

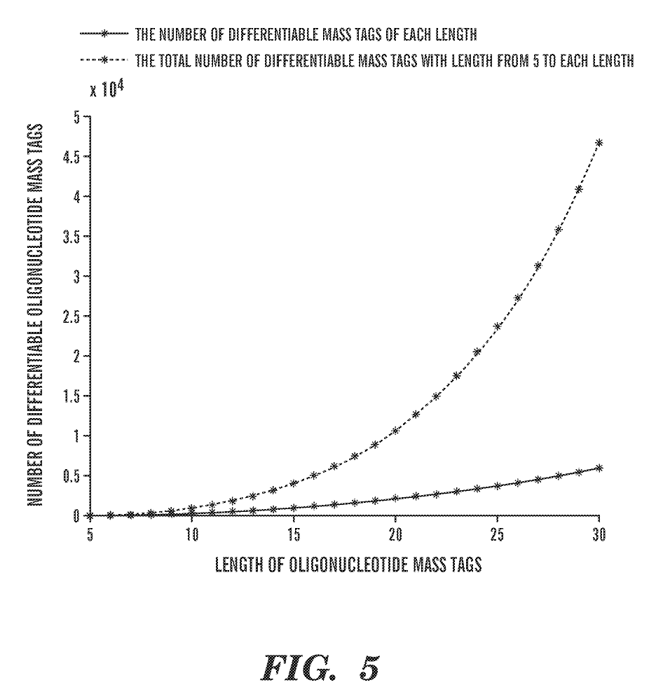

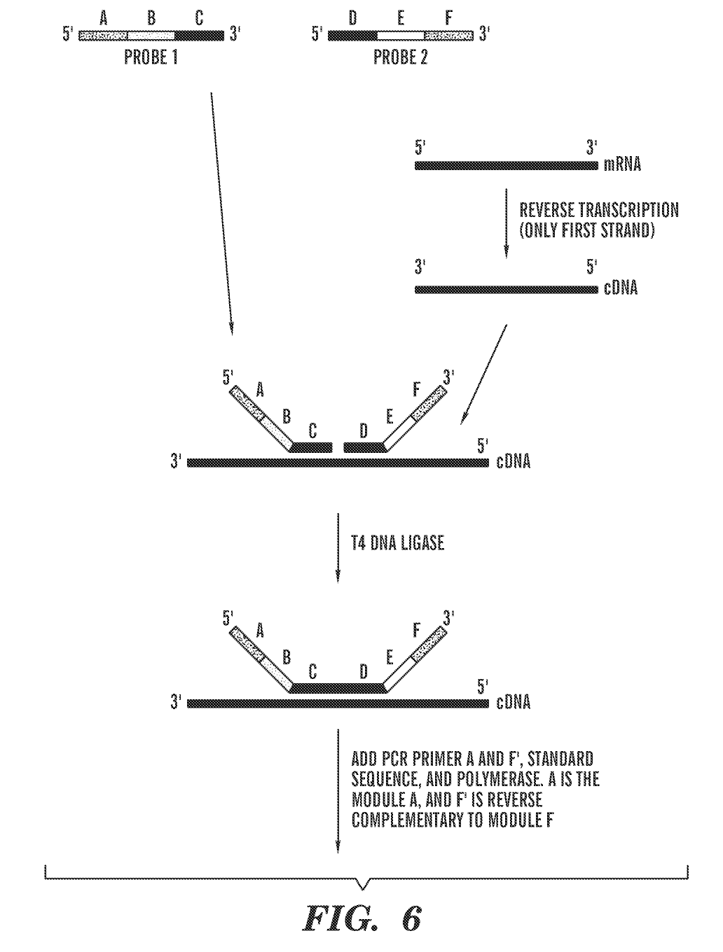

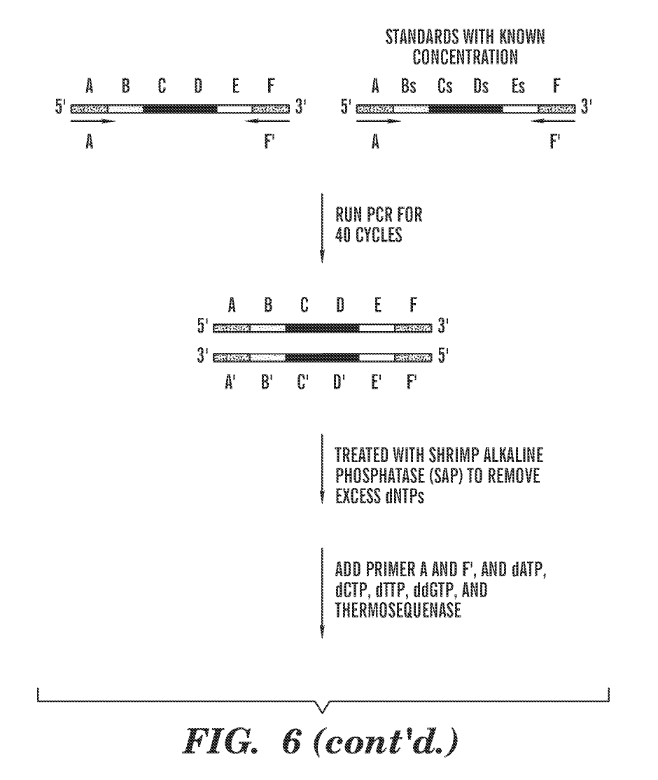

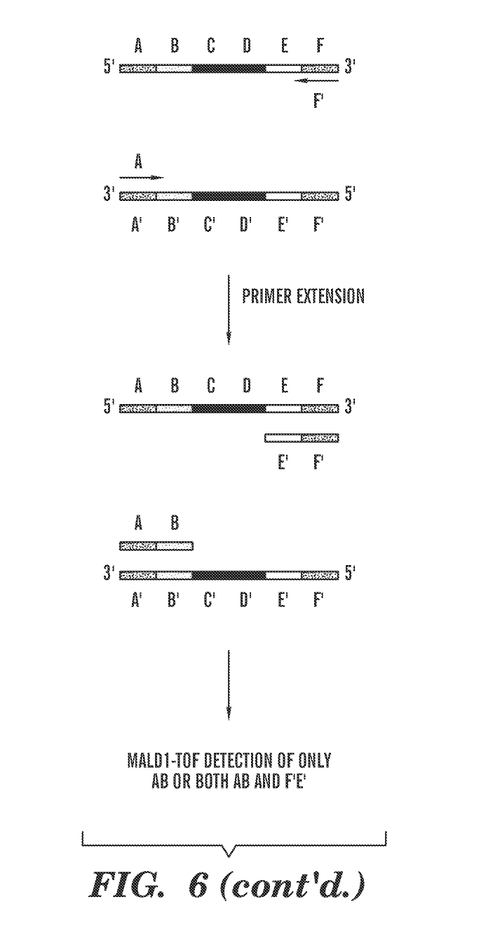

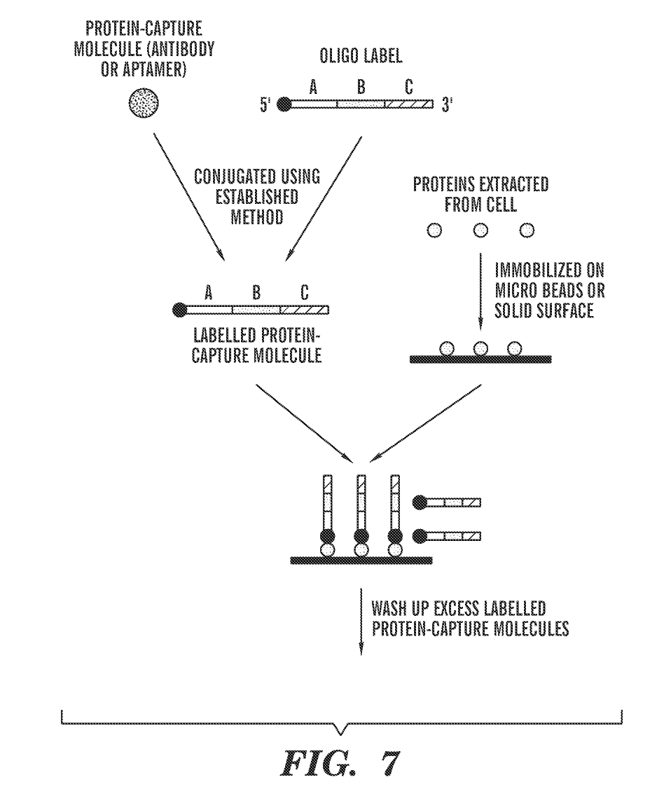

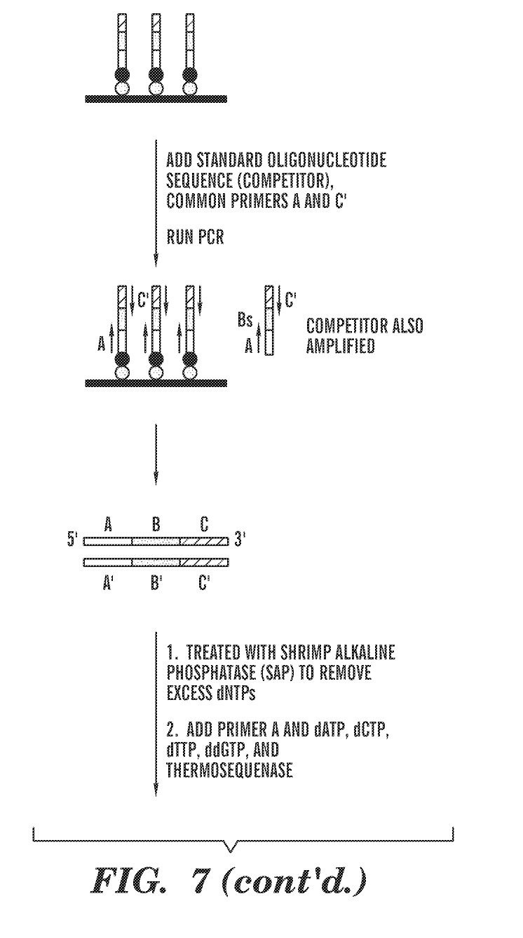

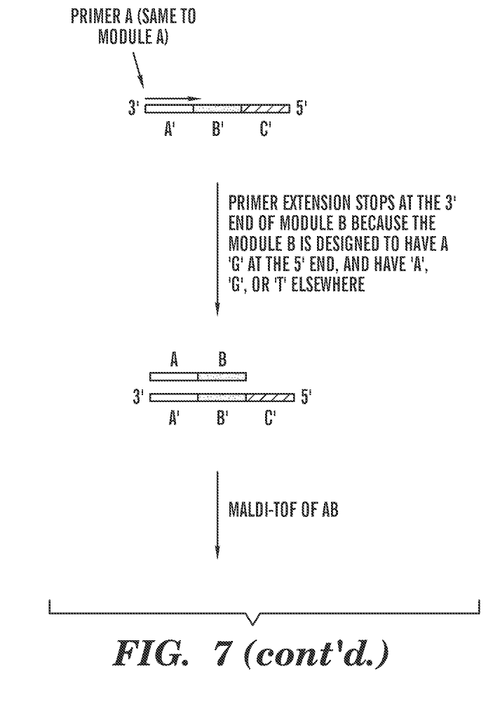

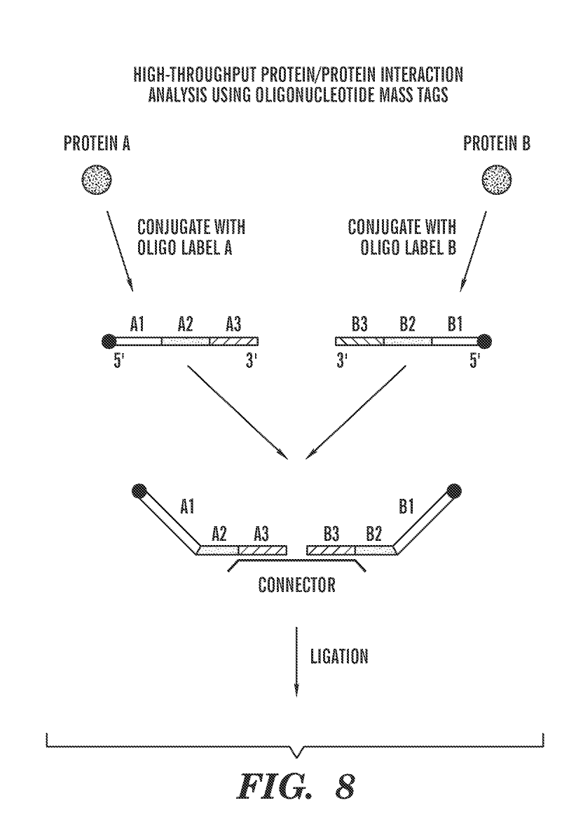

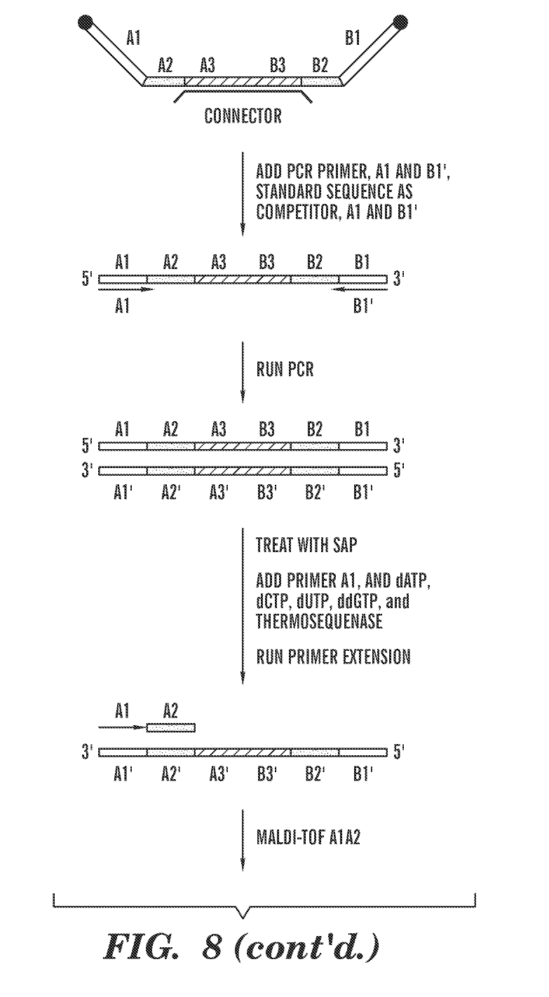

In yet another application, the invention provides a method for quantifying protein-DNA binding specificities. An example of transcription factor (TF) and double-stranded DNA binding assay, is schematically shown in FIG. 4A, where the binding specificities of a variety of sequences to the TF are quantified in a multiplexing fashion using OMTs.

For each TF of interest, a variety of probes are designed and synthesized, each of which comprises four regions, namely A, B, C, and D from 5′- to 3′-end. Region A and D, constant in all probes, are generally 15˜25 bases long and are detected by PCR primers for PCR amplifications. Region B is an OMT with a length of 1, 2, 3, 4, 5, 6, 7, 8, 9, 10, 11, 12, 13, 14, 15, 16, 17, 18, 19, 20, 21, 22, 23, 24, 25, 26, 27, 28, 29, to 30 bases. Region C comprises the candidate TF-binding sequence, which can be designed based on the prior knowledge of the consensus binding site, e.g. TRANSFAC (45), or others. Each probe is annealed with equal molar of its complementary sequences to form double-stranded probe.

Then the target TF (for example, purified TF protein or cell extract containing the target TF protein) is incubated with a mixture of equal molar of each double-stranded probes and the antibody that specifically binds the TF (1st antibody). Then a certain amount of antibody-coated beads (for example, superparamagnetic Dynabeads) is added and incubated. The antibody (2^{nd} antibody) immobilized on the bead is specific to the 1^{st} antibody (e.g. Sheep anti-Rabbit IgG). After washing up of the beads, PCR amplification is performed using PCR primers specific to region A and D and the probes immobilized on the beads serve as the templates.

After removing excessive dNTP from after the amplification reaction, for example using alkaline phosphatase, such as Shrimp alkaline Phosphatase (SAP), one adds extension primers of sequence 5′-A-3′, the proper extension mix, and a nucleic acid polymerase enzyme, such as sequenase, for example, ThermoSequenase (Sequenom). The extension mix comprises dNTP and ddNTP, where the ddNTP comprise the termination base and dNTP comprise the other three bases. Thermocycles of primer extension reaction are performed. Because of using the extension mix, the primer extension will stop at the 3′ ends of OMT (region B). At this point one gets the extension product, 5′-A-B-3′. Because region A is constant sequence and B is OMT, the mass of different extension products can be solved the mass spectroscopy and therefore the amount of each primer extension product can be quantified.

In one preferred embodiment, the final primer extension products are treated with SpectroCLEAN (Sequenom) resin to remove salts in the reaction using standard procedure. Then the reaction solution is dispensed onto a SpectroCHIP (Sequenom) prespotted with a matrix of 3-hydroxypicolinic acid (3-HPA) by using a SpectroPoint (sequenom) nanodispensor. A modified Biflex MALDI mass spectrometer was used for data acquisition from the SpectroCHIP. The quantity of each initial target can be estimated using the quantity of the extension products, 5′-A-B-3′, which is available from the mass spectroscopy using the MassTYPER software (Sequenom).

Using the above procedure, the binding specificity of TF NF-κB P50, which binds with DNA through the Rel homology domain (RHD), is analyzed. NF-κB P50 is known to co-regulate many genes and has a critical role in immunity, inflammation and apoptosis (46). Using four different probes containing the TF-binding sites of, 5′-GGGATACCCC-3′ (SEQ ID NO.: 7) (P10), 5′-GGGATATCCC (SEQ ID NO.:8) (P11), 5′-GGGGCTTCCC-3′ (SEQ ID NO.:9) (P12), 5′-GGGGCTCCCC-3′ (SEQ ID NO.:10) (P13), the binding specificity of each TF-binding site with NF-κB P50 is quantified using OMTs in one assay and the results are compared with the results from standard radioactivity-labeling gel shift analysis (46), as shown in FIG. 4B. It is shown that the two results are consistent, with a Pearson correlation coefficient of over 0.98. In addition, a 10-plex TF-DNA binding assay, which use a mixture of 10 double-stranded probes to competitively bind the target NF-κB P50, is performed and the results are compared with the gel shift analysis (46), as shown in FIG. 4C. Still the two results show strong correlation (Pearson correlation coefficient of 0.825).

Thus the method of the invention provides an OMT-based high-throughput in vitro assay of the protein-DNA binding specificities. Using the TF of NF-κB P50, it is verified that the results from multiplexing OMTs assay agree well with those from standard gel shift assay. Given the availability of genomic sequences from a large number of species, one is able to use the OMT-based high-throughput in vitro assay to quantitatively study how mutations and polymorphisms, especially the Single Nucleotide Polymorphisms (SNPs), in the candidate protein-binding sites impact the binding affinity and thus potentially affect the transcription regulation. For instance, possible TF-binding sites in a genome can be extracted from annotations or be predicted using TRANSFAC or other available tools (45). For each possible binding site containing mutation or polymorphism, e.g. SNP, an in vitro assay can be performed to check if the mutation or polymorphism potentially produces a significantly different binding affinity from the normal sequence in this position. Such piece of information will facilitate the study of many diseases related to the polymorphism-related regulation aberrations.

In one embodiment, the invention provides a method for a protein-protein interaction analysis. The protocol as shown in FIG. 3 can be used to analyze protein-protein interactions. As an alternative, using a modified protocol of protein detection using proximity-dependent DNA ligation (42), we schematically show in FIG. 4A the analysis of protein-protein interactions between proteins, A and B using oligo massTags.

For each protein, a distinct single-stranded oligonucleotide label is designed and synthesized. Each label consists of 3 regions, named from 5′ to 3′ A1, A2, and A3 for protein A and B1, B2, and B3 for protein B. Region A1 and B1 are 15˜20 bases, and A2 and B2 are about 5˜30 bases, and A3 and B3 are about 10 bases.

Protein A and B can be directly linked to their corresponding single-stranded oligonucleotide labels using established procedures (32, 40, 41). Alternatively the linkage can be achieved by introducing protein-specific aptamer at the 5′ end of each oligonucleotide label in the label synthesis step.

The labeled protein A and B are mixed together with the connector, together with a ligase, such as T4 DNA ligase, and buffer. If protein A and B interact with each other, then region A3 and B3 can be ligated together because of proximity (42).

Competitive PCR amplification: After ligation, an amplification, such as PCR primer A1 (same to region A1) and B1′ (antisense to region B1) are added and the template is amplified. In one preferred embodiment one uses PCR and performs it with about 40 cycles of PCR is run using standard protocols (18). One can naturally use fewer cycles, but using a large number of cycles typically allows one to eliminate or reduce the time required for optimization of the PCR reaction, and thus makes the detection system more robust.

After PCR, excess dNTPs are preferably removed using, for example, alkaline phosphatase, such as SAP. Primer A1 is added together with dATP, dCTP, dTTP, ddGTP, and DNA polymerase, such as THERMOSEQUENASE. The primer extension can be conducted following any standard protocols (see, e.g., 18). The primer extension will stop at the 3′ end of region A2, resulting in quantifiable products.

In one preferred embodiment, the quantification is performed using Liquid Dispensing and MALDI-TOF MS. The final primer extension products, A1A2, are treated to remove salts in the reaction buffer using standard procedure (18), for example with SpectroCLEAN (Sequenom) resin. The reaction solution is dispensed onto a SpectroCHIP (Sequenom) prespotted with a matrix of 3-hydroxypicolinic acid (3-HPA) by using a SpectroPoint (Sequenom) nanodispenser. A modified Biflex MALDI-TOF mass spectrometer (Bruker, Billerica, Mass.) was used for data acquisitions from the SpectroCHIP. The amount of sequences A1A2 can be quantitatively detected using the signal A1A2 as controls.

The amount of A1A2 reflects the amount of interacted proteins in the sample. Thus, we can estimate the amount of initial target.

### REFERENCES

The references cited herein and throughout the specification are herein incorporated by reference in their entirety.
1. Consortium, E. P. (2004) Science 306, 636-40.
2. Lander, E. S., et al. (2001) Nature 409, 860-921.
3. Pevsner, J. (2003) Bioinformatics and functional genomics (Wiley-Liss, Hoboken, N.J.).
4. Schena, M., et al., (1995) Science 270, 467-70.
5. Wang, H., et al., (2001) Genome Res 11, 1237-45.
6. Clark, T. A., et al., (2002) Science 296, 907-10.
7. Castle, J., et al., (2003) Genome Biol 4, R66.
8. Yeakley, J. M., et al., (2002) Nat Biotechnol 20, 353-8.
9. Johnson, J. M et al., (2003) Science 302, 2141-4.
10. Bertone, P., et al., (2004) Science 306, 2242-6.
11. Kapranov, P et al., (2002) Science 296, 916-9.
12. Rinn, J. L., et al., (2003) Genes Dev 17, 529-40.
13. Velculescu, V. E., et al., (1995) Science 270, 484-7.
14. Peters, D. G., et al., (1999) Nucleic Acids Res 27, e39.
15. Neilson, L., et al., (2000) Genomics 63, 13-24.
16. Velculescu, V. E, et al, (2000) Trends Genet 16, 423-5.
17. Ding, C. & Cantor, C. R. (2003) Proc Natl Acad Sci U S A 100, 3059-64.
18. Gauss, C, et al., (1999) Electrophoresis 20, 575-600.
19. Arenkov, P., et al., (2000) Anal Biochem 278, 123-31.
20. Emili, A. Q. & Cagney, G. (2000) Nat Biotechnol 18, 393-7.
21. Haab, B. B., et al., (2001) Genome Biol 2, RESEARCH0004.
22. Haab, B. B. (2001) Curr Opin Drug Discov Devel 4, 116-23.
23. Houseman, B. T et al, (2002) Nat Biotechnol 20, 270-4.
24. Huang, R. P. (2001) Clin Chem Lab Med 39, 209-14.
25. Huels, C, et al., (2002) Drug Discov Today 7, S119-24.
26. Jenison, R., et al., (2001) Clin Chem 47, 1894-900.
27. MacBeath, G. & Schreiber, S. L. (2000) Science 289, 1760-3.
28. MacBeath, G. (2002) Nat Genet 32 Suppl, 526-32.
29. Kingsmore, S. F. & Patel, D. D. (2003) Curr Opin Biotechnol 14, 74-81.
30. Stall, D., et al., (2002) Front Biosci 7, c13-32.
31. Schweitzer, B., et al., (2002) Nat Biotechnol 20, 359-65.
32. Talapatra, A., et al., (2002) Pharmacogenomics 3, 527-36.
33. Templin, M. F., et al., (2002) Drug Discov Today 7, 815-22.
34. Walter, G., et al., (2000) Curr Opin Microbiol 3, 298-302.
35. Zhu, H., et al., (2000) Nat Genet 26, 283-9.
36. Zhu, H., et al., (2001) Science 293, 2101-5.
37. Zhu, H. & Snyder, M. (2003) Curr Opin Chem Biol 7, 55-63.
38. Sano, T., et al., (1999) Nucleic Acids Res 27, 4553-61.
39. Hendrickson, E. R., et al, (1995) Nucleic Acids Res 23, 522-9.
40. Fredriksson, S., et al., (2002) Nat Biotechnol 20, 473-7.
41. Hu, G. K., Madore, S. J., Moldover, B., Jatkoe, T., Balaban, D., Thomas, J. & Wang, Y. (2001) Genome Res 11, 1237-45.
42. Mukherjee, S., Berger, M. F., Jona, G., Wang, X. S., Muzzey, D., Snyder, M., Young, R. A. & Bulyk, M. L. (2004) Nat Genet 36, 1331-9.
43. McCullough, R. M., Cantor, C. R. & Ding, C. (2005) Nucleic Acids Res 33, e99.
44. Niemeyer, C. M., Adler, M., Pignataro, B., Lenhert, S., Gao, S., Chi, L., Fuchs, H. & Blohm, D. (1999) Nucleic Acids Res 27, 4553-61.
45. Wingender, E., Dietze, P., Karas, H. & Knuppel, R. (1996) Nucleic Acids Res 24, 238-41.
46. Udalova, I. A., Mott, R., Field, D. & Kwiatkowski, D. (2002) Proc Natl Acad Sci U S A 99, 8167-72.

1. A method for detecting a target nucleic acid in a sample comprising the steps of
a) designing two 5′-3′ probes, probe 1 and probe 2, wherein
i) the 5′ end of the probe 1 comprises a region A that is a nucleic acid sequence wherein a primer 1 anneals, a region B that comprises a massTag sequence that has a known unique mass, and a region C that is the most 3′-region of the probe 1 and comprises the target recognition sequence that binds the target molecule, and
ii) the probe 2 comprises in its most 5′-region a region D that is the target recognizing sequence, then a region E that comprises a massTag, and a region F in its most 3′-region that is a constant region recognized by primer 2;
b) combining the target nucleic acid in a double-stranded form with the probe 1 and the probe 2, wherein the region C and the region D of the probes 1 and 2, respectively, anneal to the target;
c) ligating the probe 1 and the probe 2 together, wherein the ligated product consists of a continuous nucleic acid sequence comprising 5′-A-B-C-D-E-F-3″;
d) amplifying the 5′-A-B-C-D-E-F-3′ using the primer 1 and primer 2;
e) optionally removing the excess dNTPs; and
f) detecting the amplified product.

2. The method of claim 1, further comprising a step of quantifying the amount of the 5′-A-B-C-D-E-F-3′.

3. The method of claim 1, further comprising a step of quantifying the amount of the 5′-A-B-C-D-E-F-3′, wherein the quantification is performed using a primer extension reaction using a primer extension primer that anneals to either A or F region or both mid wherein the primer extension reaction is performed so that it ends at the beginning of the region C or end of the region D so as to amplify the massTag in its entirety and analyzing the primer extension products.

4. The method of claim 1, further comprising a step of quantifying the amount of the 5′-A-B-C-D-E-F-3′ wherein wherein the quantification is performed using a primer extension reaction using a primer extension primer that anneals to either A or F region or both and wherein the primer extension reaction is performed so that it ends at the beginning of the region C or end of the region D so as to amplify the massTag in its entirety arid analyzing the primer extension products the detecting in step f) is performed using mass spectrometry.

5. The method of claim 1, further comprising a step of quantifying the amount of the 5′-A-B-C-D-E-F-3′ wherein the quantification is performed using a primer extension reaction using a primer extension primer that anneals to either A or F region or both and wherein the primer extension reaction is performed so that it ends at the beginning of the region C or end of the region D so as to amplify the massTag in its entirety and analyzing the primer extension products, wherein a known amount of one or more standard sequences is added to the amplification reaction, wherein the standard sequences comprise a sequence otherwise identical to 5′-A-B-C-D-E-F-3′ except that the massTag region is designed to have a difference in its mass compared to the template 5′-A-B-C-D-E-F-3′, and wherein the primer extension reaction of the template and the standard allows determination of the absolute amount of target in the original sample.

6. A method for detecting or quantifying a target protein in a sample comprising the steps of
a) designing a probe comprising at least four regions, a region P in the most 5′-end of the probe that comprises a protein capture molecule specific to the target protein, a region A that comprises a constant nucleic acid sequence recognized by a primer 1, a region B that comprises a massTag sequence, and a region C that comprises a sequence recognized by a primer 2;
b) combining a sample suspected of containing the target protein with the P-5-A-B-C-3′-probe;
c) amplifying the 5′-A-B-C-3′ combination using the primer 1 and the primer 2;
d) optionally removing excess nucleotides;
e) performing a primer extension reaction wherein the massTag of the 5′-A-B-C-3′ is extended and results in a primer extension product if the target protein is present in the sample; and
f) detecting the primer extension product.

7. The method of claim 6, wherein a known amount of at least one standard probes otherwise identical to 5′-A-B-C-3′ except for a distinct mass Tag sequence (region B) is added to the amplification reaction, wherein the primer extension reaction of the probes and the standards allows the absolute quantification of the mass Tag sequence.

8. The method of claim 6, wherein the primer extension products is analyzed using mass spectrometry.

9. The method of claim 6, wherein the target protein is further quantified by, in addition to primer 1 and primer 2, adding to the amplification reaction a known amount of at least one standard oligonucleotide with otherwise identical sequence to 5′-A-B-C-3′ except for a different massTag, wherein after the primer extension reaction, if the target protein is present in the sample, a first primer extension product corresponding to the target protein and a second primer extension product corresponding to the standard oligonucleotide is produced, and wherein the primer extension products are detected using mass spectrometry and the ratio of the surface area that corresponds to the primer extension products with different mass indicates the amount of the target protein in the sample.

10. The method of claim 7, wherein the mass spectrometry is MALDI-TOF.

11. A method for detecting a protein-protein interaction comprising the steps of
a) designing a unique single-stranded oligonucleotide label for each of the proteins or protein fragments that are involved or suspected to be involved in the protein-protein interaction, wherein each label consists of 3 regions, named from 5′ to 3′ a region A1, a region A2, and a region A3 for first protein or protein fragment (A), and a region B1, a region B2, and a region B3 for second protein or fragment (B) and so forth, and wherein the region A1 and the region B1 are 15-20 bases long, and the region A2 and the B2 are 5-30 bases long, and the region A3 and the region B3 are 10 bases long, and so forth;
b) linking the protein or protein fragments that are involved or suspected to be involved in the protein-protein interaction to their corresponding single-stranded oligonucleotide labels;
c) mixing the labeled proteins or protein fragments that are involved or suspected to be involved in tire protein-protein interaction with a connector, together with a ligase, wherein the region A3, the region B3, arid so forth, can be ligated together only if they are in sufficient proximity which occurs if the proteins or protein fragments interact with each other; and
d) amplifying the ligated template using primer A1 that recognizes at least part of the region A1 and primer B1′ that is antisense to the region B1;
e) optionally removing excess dNTPs;
f) performing primer extension reaction, wherein the extension ends at the end of region 2 (A2 or B2′);
g) detecting the primer extension products wherein presence of a primer extension product is indicative of protein-protein interaction between the proteins or protein fragments, and wherein absence of primer extension product is indicative of no interaction between the protein or protein fragments.

12. The method of claim 10, wherein the analysis is performed using mass spectrometry.

13. The method of claim 11, further comprising adding to the amplification reaction a known amount of at least one standard probe with sequence identical to each protein probe except for the massTag sequence, wherein the primer extension reaction of the template and the standard allows determination of the absolute amount of the protein or protein fragment in the protein complex.

14. A method for analyzing DNA-protein binding specificities comprising the steps of
a) designing probes for each candidate protein-binding sequence, wherein each probe usually comprises four regions, named from 5′ to 3′ region A, comprising a constant nucleic acid sequence recognized by one PCR primer, region B, comprising the OMT sequence, region C, comprising the candidate protein-binding sequence, and region D, a constant sequence recognized by the other PCR primer;
b) annealing each probe with equal molar of its reverse complementary to form double-stranded probe;
c) incubating target protein with a mixture of double-stranded probes, and the antibody to the target protein (first antibody);
d) adding beads coated with a second antibody specific to the first antibody;
e) washing up beads and PCR amplifying probes immobilized on the beads;
f) removing excess dNTPs;
g) performing a primer extension reaction, wherein the extension products comprise 5′-A-B-3′; and
h) quantifying the primer extension products.

15. The method of claim 14, wherein the analysis is performed using mass spectroscopy.
